(19) United States
(12) Patent Application Publication
(10) Pub. No.: US 2024/0367118 A1
(43) Pub. Date: Nov. 7, 2024

(54) PRODUCTION METHOD FOR OIL-CONTAINING SILICA CAPSULE PARTICLES

(71) Applicant: KAO CORPORATION, Tokyo (JP)

(72) Inventors: Sumihiro SUZUKI, Wakayama-shi (JP); Shuichi ABE, Wakayama-shi (JP); Ryo ONISHI, Wakayama-shi (JP); Haruki IWAUCHI, Wakayama-shi (JP); Ryoichi HARUKI, Wakayama-shi (JP)

(73) Assignee: KAO CORPORATION, Tokyo (JP)

(21) Appl. No.: 18/574,174

(22) PCT Filed: Jun. 27, 2022
(86) PCT No.: PCT/JP2022/025608

§ 371 Date: Jul. 12, 2024

(30) Foreign Application Priority Data
Jun. 29, 2021 (JP) 2021-107755

(51) Int. Cl. B01F 23/41; B01J 13/02

(52) U.S. Cl. CPC B01F 23/4145; B01J 13/02

(57) ABSTRACT

The present invention relates to a method of producing oil-containing silica capsule particles having a core containing an oil and a shell containing silica as a constitutional component, including: step 1: a step of emulsifying an oil mixture liquid containing a surfactant, water, an oil, and a silica precursor, with an in-line emulsifier/disperser, so as to provide an emulsion liquid; and step 2: a step of forming oil-containing silica capsule particles by using the emulsion liquid obtained in the step 1, in a batch type agitation tank.

### FIELD OF THE INVENTION

The present invention relates to a method of producing oil-containing silica capsule particles.

### BACKGROUND OF THE INVENTION

There have been attempts to retain the effects of an oil, such as a perfume or a medical component, in such a manner that silica capsule particles containing the oil obtained by encapsulating the oil in silica capsule particles are mixed in products. In particular, one of the important capabilities of treated fiber products, cosmetics, detergents, and the like is to impart fragrance to clothes and bodies, and a product having a high capability of retaining fragrance has been demanded.

Under the circumstances, there have been studies for the synthesis of silica capsule particles containing an oil by the sol-gel method.

For example, JP 2015-128762 A (PTL 1) describes a production method of microcapsules having a first shell and a second shell each containing silica as a constitutional component, and a core containing one or more kind of an organic compound in the interior of the first shell.

JP 2017-114802 (PTL 2) describes a production method of microcapsules having a shell containing silica as a constitutional component, and a core containing polymer fine particles and one or more kind of an oil soluble liquid in the interior of the shell.

### SUMMARY OF THE INVENTION

The present invention is a method of producing oil-containing silica capsule particles having a core containing an oil and a shell containing silica as a constitutional component, and relates to a method of producing oil-containing silica capsule particles, including:
step 1: a step of emulsifying an oil mixture liquid containing a surfactant, water, an oil, and a silica precursor, with an in-line emulsifier/disperser, so as to provide an emulsion liquid; and
step 2: a step of forming oil-containing silica capsule particles by using the emulsion liquid obtained in the step 1, in a batch type agitation tank.

### DETAILED DESCRIPTION OF THE INVENTION

However, it has been found that in the case where a large amount of silica capsule particles containing an oil are produced at one time by the production methods of PTLs 1 and 2, the oil cannot be sufficiently encapsulated, and the encapsulation rate of the oil is decreased in some cases.

The present invention relates to a method of producing oil-containing silica capsule particles having a high encapsulation rate of the oil.

The present inventors have found that a method of producing oil-containing silica capsule particles having a high encapsulation rate can be provided in such a manner that in the production of silica capsule particles containing an oil, emulsification is performed with an in-line emulsifier/disperser, and silica capsule particles containing an oil are formed by using the resulting emulsion liquid in a batch type agitation tank.

Specifically, the present invention is a method of producing oil-containing silica capsule particles having a core containing an oil and a shell containing silica as a constitutional component, and relates to a method of producing oil-containing silica capsule particles, including:
step 1: a step of emulsifying an oil mixture liquid containing a surfactant, water, an oil, and a silica precursor, with an in-line emulsifier/disperser, so as to provide an emulsion liquid; and
step 2: a step of forming oil-containing silica capsule particles by using the emulsion liquid obtained in the step 1, in a batch type agitation tank.

The present invention can provide a method of producing oil-containing silica capsule particles having a high encapsulation rate of the oil.

### Method of Producing Oil-Containing Silica Capsule Particles

The production method of the present invention is a method of producing oil-containing silica capsule particles having a core containing an oil and a shell containing silica as a constitutional component (which may be hereinafter referred simply to as “silica capsule particles”), and is a method including:
step 1: a step of emulsifying an oil mixture liquid containing a surfactant, water, an oil, and a silica precursor, with an in-line emulsifier/disperser, so as to provide an emulsion liquid; and
step 2: a step of forming oil-containing silica capsule particles by using the emulsion liquid obtained in the step 1, in a batch type agitation tank.

In the present invention, the “reaction rate of a silica precursor” means the proportion of the silica precursor that at least partially reacts in the emulsion liquid. For example, in the case where a tetraalkoxysilane is used as the silica precursor, the tetraalkoxysilane that is partially hydrolyzed is deemed to have reacted, from which the reaction rate is calculated. The calculation method of the reaction rate of the silica precursor will be specifically described in the examples.

In the present invention, the “encapsulation rate of an oil” means the proportion of the oil that is encapsulated in the silica capsule particle, with respect to the amount of the oil mixed therein, and is specifically calculated by the method described in the examples.

The present invention can provide the method of producing oil-containing silica capsule particles having a high encapsulation rate of the oil. The mechanism therefor is not entirely clear, but can be considered as follows.

In the present invention, an oil mixture liquid is emulsified with an in-line emulsifier/disperser to provide an emulsion liquid in the step 1, and then the emulsion liquid is applied to sol-gel reaction in a batch type agitation tank in the step 2, so as to provide oil-containing silica capsule particles.

Examples of the installation embodiment of the in-line emulsifier/disperser used in the step 1 include an embodiment in which the in-line emulsifier/disperser is disposed outside the reservoir for reserving the oil mixture liquid. In this embodiment, the oil mixture liquid is supplied from the reservoir to the in-line emulsifier/disperser, and the oil mixture liquid can be emulsified in the emulsifying/dispersing chamber of the emulsifier/disperser. In alternative, another example of the installation embodiment of the in-line emulsifier/disperser used in the step 1 is an embodiment in which an in-line mixing unit for mixing a surfactant, water, an oil, and a silica precursor in in-line mode is provided, and the in-line emulsifier/disperser is provided on the line from the mixing unit. In this embodiment, the oil mixture liquid is supplied from the in-line mixing unit to the in-line emulsifier/disperser, the oil mixture liquid is emulsified in the emulsifying/dispersing chamber of the emulsifier/disperser, and the emulsion liquid is supplied to the reservoir. In both the embodiments, the capacity of the emulsifying/dispersing chamber in the emulsifier/disperser is smaller than the reservoir, and therefore the shearing force can be efficiently applied to the oil mixture liquid.

Due to the step 1 included, even though a large amount of the oil mixture liquid is used, it is considered that the emulsification of the oil mixture liquid can be allowed to proceed in preference to the sol-gel reaction of the silica precursor (reaction of the silica precursor), the emulsion liquid can be rapidly provided, the formation of the shell in the emulsification can be suppressed, and the breakage of the shell due to the shearing force applied thereto can be suppressed.

In the step 2, the emulsion liquid obtained in the step 1 is applied to the sol-gel reaction in the batch type agitation tank, and thereby it is considered that the partial or entire breakage of the emulsion droplets can be suppressed, the silica capsule particles having a dense and firm shell can be formed, and the encapsulation rate of the oil can be enhanced.

The “sol-gel reaction” herein means reaction in which the silica precursor forms silica as a constitutional component of the shell of the oil-containing silica capsule particles via the sol and gel states through hydrolysis and polycondensation. Examples of the sol-gel reaction include reaction in which a tetraalkoxysilane as the silica precursor is hydrolyzed, and the silanol compound forms a siloxane oligomer through dealcoholization condensation reaction and dehydration condensation reaction, followed by further progress of the dehydration condensation reaction to provide silica.

### Step 1

The step 1 is a step of emulsifying an oil mixture liquid containing a surfactant, water, an oil, and a silica precursor, with an in-line emulsifier/disperser, so as to provide an emulsion liquid.

The oil mixture liquid used in the step 1 is constituted by a water phase component containing the surfactant and water, and an oil phase component containing the oil and the silica precursor.

### Oil Mixture Liquid

### [Surfactant]

The surfactant used in the step 1 is preferably a cationic surfactant from the standpoint of enhancing the encapsulation rate of the oil.

Examples of the cationic surfactant include an alkylamine salt and an alkyl quaternary ammonium salt. The number of carbon atoms of an alkyl group of the alkylamine salt and the alkyl quaternary ammonium salt is preferably 10 or more, more preferably 12 or more, and further preferably 14 or more, and is preferably 22 or less, more preferably 20 or less, and further preferably 18 or less.

Examples of the alkylamine salt include an alkylamine acetate, such as laurylamine acetate and stearylamine acetate.

Examples of the alkyl quaternary ammonium salt include an alkyltrimethyl ammonium salt, a dialkyldimethyl ammonium salt, and an alkylbenzyldimethyl ammonium salt.

Examples of the alkyltrimethyl ammonium salt include an alkyltrimethyl ammonium chloride, such as lauryltrimethyl ammonium chloride, cetyltrimethyl ammonium chloride, and stearyltrimethyl ammonium chloride, and an alkyltrimethyl ammonium bromide, such as lauryltrimethyl ammonium bromide, cetyltrimethyl ammonium bromide, and stearyltrimethyl ammonium bromide.

Examples of the dialkyldimethyl ammonium salt include a dialkyldimethyl ammonium chloride, such as distearyldimethyl ammonium chloride, and a dialkyldimethyl ammonium bromide, such as distearyldimethyl ammonium bromide.

Examples of the alkylbenzyldimethyl ammonium salt include an alkylbenzyldimethyl ammonium chloride and an alkylbenzyldimethyl ammonium bromide.

One kind of the cationic surfactant may be used alone, or two or more kinds thereof may be used.

Among the above, the cationic surfactant is preferably a quaternary ammonium salt, more preferably an alkyltrimethyl ammonium salt having an alkyl group having 10 or more and 22 or less carbon atoms, further preferably one or more kind selected from lauryltrimethyl ammonium chloride, stearyltrimethyl ammonium chloride, and cetyltrimethyl ammonium chloride, and still further preferably cetyltrimethyl ammonium chloride.

The amount of the surfactant used in the step 1 is preferably 0.1 part by mass or more, more preferably 0.2 part by mass or more, and further preferably 0.3 part by mass or more, and is preferably 5 parts by mass or less, more preferably 3 parts by mass or less, further preferably 1 part by mass or less, and still further preferably 0.7 part by mass or less, per 100 parts by mass of the oil used in the step 1, from the standpoint of providing a stable emulsion liquid.

### [Water]

The water used in the step 1 is preferably, for example, one or more kind selected from ion exchanged water and distilled water.

The amount of the water used in the step 1 is preferably 100 parts by mass or more, more preferably 130 parts by mass or more, and further preferably 150 parts by mass or more, and is preferably 300 parts by mass or less, more preferably 250 parts by mass or less, and further preferably 200 parts by mass or less, per 100 parts by mass of the oil used in the step 1, from the standpoint of providing a stable emulsion liquid.

### Oil

The oil used in the step 1 becomes an encapsulated component of the resulting silica capsule particles.

The oil is preferably one or more kind selected from a perfume, a perfume precursor, a moisturizing agent, an antioxidant, an antibacterial agent, a fertilizer, a surface modifier for fibers, skin, hair, and the like, a cooling agent, a dye, a colorant, a silicone, and an oil soluble polymer, more preferably one or more kind selected from a perfume, a perfume precursor, a moisturizing agent, an antioxidant, an antibacterial agent, a fertilizer, and a surface modifier, further preferably one or more kind selected from a perfume, a perfume precursor, a moisturizing agent, and an antioxidant, still further preferably one or more kind selected from a perfume, a perfume precursor, and a moisturizing agent, and still more further preferably one or more kind selected from a perfume and a perfume precursor.

One kind of the oil may be used alone, or two or more kinds thereof may be used.

Examples of the perfume precursor include a compound releasing a perfume component through reaction with water, and a compound releasing a perfume component through reaction with light.

Examples of the compound releasing a perfume component through reaction with water include a silicate ester compound having an alkoxy component derived from a perfume alcohol, an fatty acid ester compound having an alkoxy component derived from a perfume alcohol, an acetal compound or a hemiacetal compound obtained through reaction of a carbonyl component derived from a perfume aldehyde or a perfume ketone and an alcohol compound, a Schiff base compound obtained through reaction of a carbonyl component derived from a perfume aldehyde or a perfume ketone and a primary amine compound, and a hemiaminal compound or a hydrazone compound obtained through reaction of a carbonyl component derived from a perfume aldehyde or a perfume ketone and a hydrazine compound.

Examples of the compound releasing a perfume component through reaction with light include a 2-nitrobenzyl ether compound having an alkoxy component derived from a perfume alcohol, an α-keto ester compound having a carbonyl component derived from a perfume aldehyde or a perfume ketone, and a coumarate ester compound having an alkoxy component derived from a perfume alcohol. These perfume precursors each may be, for example, a polymer, such as a reaction product of a part of a carboxy group of a polyacrylic acid and a perfume alcohol.

The calculated value of common logarithm of the distribution coefficient P of the oil between n-octanol and water (n-octanol/water) LogP (which may be hereinafter referred to as a “cLogP value”) is preferably 1 or more, more preferably 2 or more, and further preferably 3 or more, and is preferably 30 or less, more preferably 20 or less, and further preferably 10 or less, from the standpoint of providing a stable emulsion liquid.

In the case where the oil is constituted by multiple kinds of constitutional components, the cLogP value of the oil can be calculated in such a manner that the cLogP values of the constitutional components are multiplied by the volume proportions thereof respectively, and the resulting values are summated.

With a cLogP value of the oil of 1 or more, the encapsulation rate of the oil in the silica capsule particles obtained in the sol-gel reaction of the silica precursor can be enhanced. Even in the case where the oil is a perfume composition constituted by multiple perfume components, with a cLogP value of the perfume composition of 1 or more, the encapsulation rate of the perfume composition in the resulting silica capsule particles can be enhanced.

The cLogP value herein is the “Log P (cLogP)” calculated by the method described in A. Leo Comprehensive Medicinal Chemistry, Vol. 4 C. Hansch, P.G. Sammens, J.B. Taylor and C.A. Ramsden, Eds., P. 295, Pergamon Press, 1990, and a cLogP value calculated with a program, CLOGP v. 4.01, may be used.

### [Silica Precursor]

The silica precursor used in the step 1 preferably contains a tetraalkoxysilane, and is more preferably a tetraalkoxysilane having an alkoxy group having 1 or more and 4 or less carbon atoms, further preferably one or more kind selected from tetramethoxysilane, tetraethoxysilane, and tetraisopropoxysilane, still further preferably one or more kind selected from tetramethoxysilane and tetraethoxysilane, and still more further preferably tetraethoxysilane, from the standpoint of allowing the emulsification rapidly and enhancing the encapsulation rate of the oil while suppressing the reaction of the silica precursor in the step 1.

In the case where the silica precursor contains a tetraalkoxysilane, a trialkoxysilane, such as triethoxysilane and trimethoxysilane, may be contained, and the content of the tetraalkoxysilane in the silica precursor is preferably 80% by mass or more, more preferably 85% by mass or more, and further preferably 90% by mass or more, and is preferably 100% by mass or less.

The amount of the silica precursor used in the step 1 is preferably 10 parts by mass or more, more preferably 15 parts by mass or more, and further preferably 20 parts by mass or more, from the standpoint of forming a shell capable of surrounding the emulsion droplets containing the oil, and is preferably 100 parts by mass or less, more preferably 70 parts by mass or less, further preferably 50 parts by mass or less, still further preferably 40 parts by mass or less, and still more further preferably 30 parts by mass or less, from the standpoint of suppressing the silica precursor from remaining inside the emulsion droplets and facilitating the conversion thereof to the shell in the sol-gel reaction, all per 100 parts by mass of the oil used in the step 1.

The oil mixture liquid used in the step 1 may contain an emulsification aid, a particle diameter stabilizer, and the like, as an oil phase component other than the oil and the silica precursor.

### [Emulsification Aid]

Preferred examples of the emulsification aid include one or more kind selected from a higher aliphatic alcohol having 6 or more carbon atoms, a higher fatty acid having 6 or more carbon atoms, a monoalkyl glyceryl ether having an alkyl group having 6 or more carbon atoms, and an amide compound having an alkyl group having 8 or more carbon atoms.

It is considered that the emulsification aid can allow the emulsification to proceed rapidly and can enhance the encapsulation rate of the oil while suppressing the reaction of the silica precursor in the emulsification of the oil mixture liquid in the step 1, due to the long-chain aliphatic hydrocarbon group and the polar group thereof.

One kind of the emulsification aid may be used alone, or two or more kinds thereof may be used.

The molecular weight of the emulsification aid is preferably 500 or less, more preferably 450 or less, further preferably 400 or less, and still further preferably 350 or less, and is preferably 150 or more, from the standpoint of allowing the emulsification to proceed rapidly and enhancing the encapsulation rate of the oil while suppressing the reaction of silica precursor.

The number of carbon atoms of the higher aliphatic alcohol is preferably 8 or more, more preferably 10 or more, further preferably 12 or more, and still further preferably 14 or more, and is preferably 22 or less, more preferably 20 or less, and further preferably 18 or less, from the standpoint of allowing the emulsification to proceed rapidly and enhancing the encapsulation rate of the oil while suppressing the reaction of silica precursor.

The higher aliphatic alcohol is preferably a linear or branched higher aliphatic alcohol, and more preferably a linear higher aliphatic primary alcohol, from the same standpoint as above.

The higher aliphatic alcohol is preferably in a solid state under ordinary temperature and ordinary pressure (for example, having a melting point of 30° C. or more) from the standpoint of the handleability. The melting point of the higher aliphatic alcohol is preferably 30° C. or more, more preferably 35° C. or more, further preferably 40° C. or more, and still further preferably 45° C. or more.

Examples of the higher aliphatic primary alcohol include 2-ethy lhexyl alcohol, lauryl alcohol, myristyl alcohol, cetyl alcohol, stearyl alcohol, behenyl alcohol, and oleyl alcohol. Among these, one or more kind selected from 2-ethylhexyl alcohol, lauryl alcohol, myristyl alcohol, cetyl alcohol, and stearyl alcohol is preferred, one or more kind selected from cetyl alcohol and stearyl alcohol is more preferred, and cetyl alcohol is further preferred.

The number of carbon atoms of the higher fatty acid is preferably 8 or more, more preferably 10 or more, further preferably 12 or more, still further preferably 14 or more, and still more further preferably 16 or more, and is preferably 26 or less, more preferably 22 or less, and further preferably 20 or less, from the standpoint of allowing the emulsification to proceed rapidly and enhancing the encapsulation rate of the oil while suppressing the reaction of silica precursor.

Examples of the higher fatty acid include 2-ethylhexanoic acid, lauric acid, myristic acid, palmitic acid, stearic acid, behenic acid, oleic acid, linoleic acid, linolenic acid, lanolin acid, and isostearic acid. Among these, a branched saturated fatty acid is preferred, and isostearic acid is more preferred.

The number of carbon atoms of the alkyl group of the monoalkyl glyceryl ether is preferably 10 or more, more preferably 12 or more, further preferably 14 or more, and still further preferably 16 or more, and is preferably 24 or less, more preferably 22 or less, and further preferably 20 or less, from the standpoint of allowing the emulsification to proceed rapidly and enhancing the encapsulation rate of the oil while suppressing the reaction of silica precursor.

Examples of the monoalkyl glyceryl ether include mono-2-ethylhexyl glyceryl ether, monodecyl glyceryl ether, monolauryl glyceryl ether, monomyristyl glyceryl ether, monocetyl glyceryl ether, monostearyl glyceryl ether, and monobehenyl glyceryl ether. Among these, one or more kind selected from monocetyl glyceryl ether, monostearyl glyceryl ether, and monobehenyl glyceryl ether is preferred, and monostearyl glyceryl ether is more preferred. The monoalkyl glyceryl ether is generally an α-isomer.

The number of carbon atoms of the alkyl group of the amide compound is preferably 10 or more, more preferably 12 or more, and further preferably 14 or more, and is preferably 22 or less, more preferably 20 or less, and further preferably 18 or less, from the standpoint of allowing the emulsification to proceed rapidly and enhancing the encapsulation rate of the oil while suppressing the reaction of silica precursor.

The amide compound is preferably an amide compound having an alkyl group derived from a saturated or unsaturated fatty acid. Specific examples thereof include lauric acid amide, myristic acid amide, palmitic acid amide, stearic acid amide, and oleic acid amide.

The cLogP value of the emulsification aid is preferably 4 or more, more preferably 5 or more, and further preferably 6 or more, and is preferably 10 or less, and more preferably 9 or less, from the standpoint of allowing the emulsification to proceed rapidly and enhancing the encapsulation rate of the oil while suppressing the reaction of silica precursor.

It is considered that the higher fatty acid and the higher aliphatic alcohol have a function of a particle diameter stabilizer stabilizing the particle diameter of the emulsion droplets, in addition to the function of an emulsification aid. From this standpoint, the oil mixture liquid used in the step 1 preferably contains, as the oil phase component other than the oil and the silica precursor, one or more kind selected from a higher fatty acid having 6 or more carbon atoms and a higher aliphatic alcohol having 6 or more carbon atoms, and more preferably contains a higher fatty acid having 6 or more carbon atoms.

In the case where the oil mixture liquid used in the step 1 further contains the emulsification aid the oil phase component, the amount of the emulsification aid is preferably 0.01 part by mass or more, more preferably 0.1 part by mass or more, and further preferably 0.5 part by mass or more, and is preferably 10 parts by mass or less, more preferably 7 parts by mass or less, and further preferably 5 parts by mass or less, per 100 parts by mass of the oil used in the step 1, from the standpoint of allowing the emulsification to proceed rapidly and enhancing the encapsulation rate of the oil while suppressing the reaction of silica precursor.

### [Particle Diameter Stabilizer]

Examples of the particle diameter stabilizer include a fatty acid ester having 6 or more carbon atoms in total.

The particle diameter stabilizer contributes to the suppression of the destabilization of the emulsion droplets through Ostwald ripening, in which a relatively hydrophilic component in the oil phase component in the emulsion droplets undergoes molecular diffusion to the water phase as the continuous phase, and thereby it is considered that the emulsion droplets can be suppressed from becoming coarse with the lapse of time, and the particle diameter of the emulsion droplets can be stabilized. Accordingly, it is considered that the combination use of the particle diameter stabilizer and the emulsification aid can allow the emulsification to proceed rapidly while suppressing the reaction of the silica precursor, providing an environment suitable for forming a shell as a mold of the silica capsule particles, and can enhance the encapsulation rate of the oil. From this standpoint, the cLogP value of the particle diameter stabilizer is preferably 4 or more, more preferably 5 or more, further preferably 6 or more, and still further preferably 7 or more, and is preferably 10 or less, and more preferably 9 or less.

The total number of carbon atoms of the fatty acid ester is preferably 10 or more, more preferably 14 or more, and further preferably 18 or more, and is preferably 50 or less, from the standpoint of allowing the emulsification to proceed rapidly and enhancing the encapsulation rate of the oil while suppressing the reaction of silica precursor.

Examples of the fatty acid ester include a fatty acid monoester of a fatty acid and a monohydric alcohol, a fatty acid diester of a fatty acid and a dihydric alcohol, a dicarboxylate diester of a dicarboxylic acid and a monohydric alcohol, a tricarboxylate triester of a tricarboxylic acid and a monohydric alcohol, and a glycerin fatty acid triester. Among these, a fatty acid monoester is preferred from the standpoint of allowing the emulsification to proceed rapidly and enhancing the encapsulation rate of the oil while suppressing the reaction of silica precursor.

The fatty acid monoester is preferably formed of a fatty acid having 8 or more and 22 or less carbon atoms and a monohydric alcohol having 1 or more and 24 or less carbon atoms.

Examples of the fatty acid constituting the fatty acid monoester include a saturated or unsaturated fatty acid having 8 or more and 22 or less carbon atoms, such as 2-ethylhexanoic acid, lauric acid, myristic acid, pentadecanoic acid, palmitic acid, palmitoleic acid, margaric acid, stearic acid, oleic acid, linoleic acid, erucic acid, arachidic acid, and behenic acid.

Examples of the monohydric alcohol constituting the fatty acid monoester include a monohydric alcohol having 1 or more and 24 or less carbon atoms, such as methanol, ethanol, n-propyl alcohol, isopropyl alcohol, n-butyl alcohol, isobutyl alcohol, n-pentyl alcohol, isopentyl alcohol, neopentyl alcohol, hexanol, heptanol, octanol, 2-ethylhexyl alcohol, nonanol, isononyl alcohol, decanol, isodecyl alcohol, dodecanol, lauryl alcohol, tridecanol, myristyl alcohol, pentadecanol, cetyl alcohol, stearyl alcohol, isostearyl alcohol, oleyl alcohol, behenyl alcohol, and 2-octyldodecanol.

Examples of the fatty acid monoester include cetyl 2-ethylhexanoate, butyl stearate, isopropyl myristate, hexadecyl myristate, 2-octyldodecyl myristate, isopropyl palmitate, hexadecyl palmitate, and 2-ethy lhexyl stearate. Among these, isopropyl palmitate is preferred.

In the case where the oil mixture liquid used in the step 1 further contains the particle diameter stabilizer as the oil phase component, the amount of the particle diameter stabilizer is preferably 0.01 part by mass or more, more preferably 0.1 part by mass or more, and further preferably 0.5 part by mass or more, and is preferably 10 parts by mass or less, more preferably 7 parts by mass or less, and further preferably 5 parts by mass or less, per 100 parts by mass of the oil used in the step 1, from the standpoint of allowing the emulsification to proceed rapidly and enhancing the encapsulation rate of the oil while suppressing the reaction of silica precursor.

The mass ratio of the water phase component containing the surfactant and water and the oil phase component containing the oil and the silica precursor of the oil mixture liquid used in the step 1 (water phase component/oil phase component) is preferably 50/50 or more, more preferably 53/47 or more, and further preferably 57/43 or more, from the standpoint of achieving the stable emulsion liquid, and is preferably 99/1 or less, more preferably 95/5 or less, further preferably 90/10 or less, still further preferably 80/20 or less, and still more further preferably 70/30 or less, from the standpoint of the production efficiency.

### Emulsification of Oil Mixture Liquid

Preferred examples of the emulsification method in the step 1 include a method in which the water phase component and the oil phase component, which have been prepared in advance, are mixed in the reservoir to prepare the oil mixture liquid, and then the oil mixture liquid is supplied to the in-line emulsifier/disperser provided outside the reservoir for emulsification. In the preparation of the oil mixture liquid of this method, the order of charging the water phase component and the oil phase component to the reservoir is not particularly limited, and it is preferred that the water phase component is prepared in the reservoir, and then the oil phase component having been prepared separately is added thereto, from the standpoint of the easiness in production.

The reservoir is preferably equipped with an agitation device having an agitation blade.

Examples of the agitation blade include a paddle blade, a turbine blade, an anchor blade, a ribbon blade, and a propeller. In this case, the batch type agitation tank used in the step 2 is preferably used as the reservoir from the standpoint of the easiness in production.

Preferred examples of the emulsification method in the step 1 also include a method in which the surfactant, water, the oil, and the silica precursor are mixed in in-line mode to prepare the oil mixture liquid, and then the oil mixture liquid is supplied to the in-line emulsifier/disperser for emulsification. In the case where oil mixture liquid is prepared by mixing in in-line mode, it is preferred that the water phase component containing the surfactant and water and the oil phase component containing the oil and the silica precursor are mixed in a single line (inside piping) to prepare the oil mixture liquid.

The water phase component applied to the in-line mixing is prepared preferably by using an agitation device having an agitation blade. Examples of the agitation blade include the same ones as in the agitation blade used in the reservoir. The oil phase component applied to the in-line mixing may be prepared by using an agitation device having an agitation blade, or may be prepared by mixing the oil and the silica precursor in in-line mode.

The in-line mixing is preferably performed by using an in-line mixer, such as a static mixer.

In the case where the water phase component is prepared by using the agitation device, the rotation number in agitation in the preparation of the water phase component is preferably 20 r/min or more, more preferably 30 r/min or more, and further preferably 50 r/min or more, in consideration of the tip peripheral speed, and is preferably 120 r/min or less, more preferably 100 r/min or less, and further preferably 90 r/min or less, from the standpoint of suppressing temperature rise due to agitation.

The “tip peripheral speed” in the present invention means, in the case where an agitation blade is used, the peripheral speed of the outer periphery of the largest agitation blade (main agitation blade) in the agitation device.

The liquid temperature in agitation in the preparation of the water phase component using the agitation device is preferably 0° C. or more, and is preferably 40° C. or less, and more preferably 35° C. or less.

The agitation time in the preparation of the water phase component using the agitation device is preferably 3 minutes or more, more preferably 5 minutes or more, and further preferably 10 minutes or more, and is preferably 60 minutes or less, more preferably 30 minutes or less, and further preferably 20 minutes or less, while depending on the production scale, the agitation speed, the temperature condition, and the like.

The amount of the oil mixture liquid used in the step 1 is preferably 10 kg or more, more preferably 100 kg or more, further preferably 300 kg or more, still further preferably 500 kg or more, and still more further preferably 1,000 kg or more, from the standpoint of the productivity, and is preferably 100,000 kg or less, and more preferably 10,000 kg or less, from the standpoint of the equipments.

In the present invention, the large amount of the oil mixture liquid means that the amount of the oil mixture liquid used in the step 1 is 100 kg or more.

In the case where the oil mixture liquid is prepared in the reservoir, it is preferred that in the step 1, the oil mixture liquid in the reservoir is supplied to the in-line emulsifier/disperser while circulating and mixing with the agitation blade. The rotation number in agitation in this case is preferably 20 r/min or more, more preferably 30 r/min or more, and further preferably 50 r/min or more, in consideration of the tip peripheral speed, and is preferably 120 r/min or less, more preferably 100 r/min or less, and further preferably 90 r/min or less, from the standpoint of suppressing temperature rise due to agitation.

In the case where the oil mixture liquid is prepared in the reservoir, the oil mixture liquid used in the step 1 may be applied to preliminary emulsification with the agitation blade before supplying to the in-line emulsifier/disperser. The preferred range of the rotation number in agitation in this case is the same as the preferred range of the rotation number in agitation in the circulation and mixing described above.

The liquid temperature of the oil mixture liquid applied to the preliminary emulsification is preferably 0° C. or more, and is preferably 40° C. or less, and more preferably 35° C. or less.

The agitation time in the preliminary emulsification is preferably 3 minutes or more, more preferably 5 minutes or more, and further preferably 10 minutes or more, and is preferably 60 minutes or less, more preferably 30 minutes or less, and further preferably 20 minutes or less, while depending on the production scale, the agitation speed, the temperature condition, and the like.

The emulsification in the step 1 is performed by using the in-line emulsifier/disperser from the standpoint of allowing the emulsification to proceed rapidly and enhancing the encapsulation rate of the oil while suppressing the reaction of silica precursor.

The emulsification in the step 1 may be a “one-pass system” in which the oil mixture liquid is allowed to pass through the in-line emulsifier/disperser only once, or may be a “multi-pass system” in which a part or the entire of the emulsion liquid obtained by allowing the oil mixture liquid to pass through the in-line emulsifier/disperser is again allowed to pass through the in-line emulsifier/disperser, and the operation is performed multiple times.

Examples of the system of the one-pass system include an in-line system.

Examples of the system of the multi-pass system include an “external circulation system”, a “back-and-forth system”, a “liquid return system”, and an “in-line system”.

The external circulation system is a method in which the oil mixture liquid is discharged from the reservoir equipped with an external circulation line connected to the in-line emulsifier/disperser, via the external circulation line, and emulsified with the in-line emulsifier/disperser, and then the emulsion liquid is returned to the reservoir via the external circulation line, so that the emulsion liquid is circulated between the in-line emulsifier/disperser and the reservoir.

The back-and-forth system is a method in which a line connected to the in-line emulsifier/disperser is provided between two reservoirs, and the emulsion liquid is allowed to reciprocate between the reservoirs via the line.

The liquid return system is a method in which with two reservoirs and the in-line emulsifier/disperser connected via a line to enable circulation of the liquid among them, the entire amount of the liquid is allowed to pass through the in-line emulsifier/disperser, and then returned to the original reservoir, and the operation is repeated.

Preferred examples of the in-line system include a method in which the surfactant, water, the oil, and the silica precursor are mixed in in-line mode to prepare the oil mixture liquid, and then the oil mixture liquid is supplied to the in-line emulsifier/disperser and emulsified with the in-line emulsifier/disperser.

Among the systems, the emulsification in the step 1 is preferably performed by the external circulation system or the in-line system using the in-line emulsifier/disperser from the standpoint of allowing the emulsification to proceed rapidly and enhancing the encapsulation rate of the oil while suppressing the reaction of silica precursor and the standpoint of the easiness in designing the equipments.

In the external circulation system, it is preferred that the emulsion liquid is circulated between the in-line emulsifier/disperser and one reservoir via the external circulation line from the standpoint of allowing the emulsification to proceed rapidly and enhancing the encapsulation rate of the oil while suppressing the reaction of silica precursor and the standpoint of the easiness in designing the equipments.

Even in the external circulation system, it is possible that the oil mixture liquid is discharged from the reservoir via the external circulation line and emulsified with the in-line emulsifier/disperser, and then returned to the same reservoir as the original reservoir via the external circulation line, and also it is possible that the liquid is delivered to the other reservoir than the original reservoir via a line.

In the in-line system, it is preferred that the in-line emulsifier/disperser is attached to the line from the in-line mixing unit for mixing the surfactant, water, the oil, and the silica precursor in in-line mode, and the materials are emulsified with the in-line emulsifier/disperser.

In the in-line system, it suffices that the oil mixture liquid obtained by mixing in in-line mode is allowed to pass through the in-line emulsifier/disperser once or more. In other words, in the in-line system, the number of pass may be one (i.e., a one-pass system) or multiple passes (i.e., a multi-pass system).

In the in-line system, in the case where the oil mixture liquid is allowed to pass through the in-line emulsifier/disperser multiple times, it is possible that the emulsion liquid obtained by allowing the oil mixture liquid discharged from the in-line mixing unit to pass through the in-line emulsifier/disperser is reserved in a reservoir, and by connecting the reservoir and the in-line emulsifier/disperser via a line for circulating the liquid between them, at least a part of the entire of the emulsion liquid is allowed to pass through the in-line emulsifier/disperser.

In any of the aforementioned systems, two or more in-line emulsifier/dispersers may be connected to each other.

The in-line emulsifier/disperser preferably has a rotor and a stator from the standpoint of applying a shearing force to the oil mixture liquid efficiently, allowing the emulsification to proceed rapidly, and enhancing the encapsulation rate of the oil, while suppressing the reaction of silica precursor.

The emulsifier/disperser having a rotor and a stator may be an optional disperser using a shearing field generated between the rotor and the stator in the emulsifying/dispersing chamber. Specifically, the shearing speed is regulated by changing variously the clearance between the rotor and the stator and the rotation number of the rotor, and the oil mixture liquid is allowed to pass between the rotating rotor and the stator in the emulsifying/dispersing chamber, thereby applying the shearing force to the oil mixture liquid for emulsification.

Examples of the in-line emulsifier/disperser include Cavitron (available from Eurotec Co., Ltd.), Milder (available from Pacific Machinery & Engineering Co., Ltd.), and the like. Preferred examples among these include Cavitron (available from Eurotec Co., Ltd.) and Milder (available from Pacific Machinery & Engineering Co., Ltd.), and particularly preferred examples thereof include Cavitron (available from Eurotec Co., Ltd.), from the standpoint of enabling application of the desired shearing force to the oil mixture liquid.

The shearing force applied to the oil mixture liquid in the step 1 is preferably regulated by using the rotation energy Q of the rotor per the capacity of the emulsifying/dispersing chamber of the in-line emulsifier/disperser as an index.

The rotation energy Q of the rotor per the capacity of the emulsifying/dispersing chamber of the in-line emulsifier/disperser can be obtained according to the following calculation expression (I).

$$Rotation energy Q ( W / {m}^{3} ) = ( required rotation power of rotor P ( W ) ) / ( capacity of emulsifying / dispersing chamber of in - line emulsifer / disperser ( {m}^{3} ) ) \qquad ( I )$$

In the expression (I), the required rotation power P of the rotor (W) is calculated from the following (experimental expression 1).

$$Required rotation power P of the rotor ( W ) = {n}^{3} \times {d}^{5} \times ρ \qquad ( experimental expression 1 )$$

n: rotation number of rotor (s⁻¹)
d: outer diameter of rotor (m)
ρ: density of oil mixture liquid (kg/m³)

As for the value of ρ, the approximate value of 1,000 (kg/m³) is used.

The rotation energy Q is preferably 1×10⁷ W/m³ or more, more preferably 1×10⁸ W/m³ or more, and further preferably 1×10⁹ W/m³ or more, from the standpoint of enhancing the shearing effect on the oil mixture liquid and producing the fine emulsion liquid having an average particle diameter of 10 μm or less rapidly while suppressing the reaction rate of the silica precursor. The rotation energy Q may be preferably 1×10¹² W/m³ or less from the standpoint of the production efficiency.

The shearing force applied to the oil mixture liquid in the emulsification of the step 1 may also be regulated by using the rotation energy Q′ from the rotation energy Q of the rotor per the capacity of the emulsifying/dispersing chamber of the in-line emulsifier/disperser in consideration of the emulsification time as an index.

The rotation energy Q′ can be obtained according to the following calculation expression (II).

$$Rotation energy {Q}^{′} ( W \cdot h / {m}^{3} ) = ( ( required rotation power of rotor P ( W ) ) \times ( emulsification time ( h ) ) ) / ( capacity of emulsifying / dispering chamber of in - line emulsifier / disperser ( {m}^{3} ) ) \qquad ( II )$$

In the expression (II), the required rotation power of rotor P (W), n, d, and ρ are the same as in the expression (I).

As for the value of ρ, the approximate value of 1,000 (kg/m³) is used.

The rotation energy Q′ is preferably 1×10⁶ W·h/m³ or more, more preferably 1×10⁷ W·h/m³ or more, and further preferably 1×108 W·h/m³ or more, from the standpoint of enhancing the shearing effect on the oil mixture liquid and producing the fine emulsion liquid having an average particle diameter of 10 μm or less rapidly while suppressing the reaction rate of the silica precursor. The rotation energy Q′ may be preferably 1×10¹² W·h/m³ or less from the standpoint of the production efficiency.

In the case where multiple in-line emulsifier/dispersers are used in series, or in the case where the in-line emulsifier/disperser having multiple stages each including a rotor and a stator in the emulsifying/dispersing chamber, such as Milder (available from Pacific Machinery & Engineering Co., Ltd.), is used, the required rotation power of rotor P (W) means the total of the required rotation power of rotor P (W) calculated for each of the in-line emulsifier/dispersers, or the total of the required rotation power of rotor P (W) calculated for each of the stages.

In the case where the conditions, such as the rotation number of the rotor, are changed during the emulsification in the step 1, the rotation energy Q′ (W·h/m³) is obtained as the total of the rotation energy Q′ (W·h/m³) calculated from each of the conditions and the emulsification time in that condition.

The rotation energy Q′ (W·h/m³) per the amount (kg) of the oil mixture liquid obtained by dividing rotation energy Q′ (W·h/m³) by the amount (kg) of the oil mixture liquid used in the step 1 (i.e., the rotation energy Q″ (W·h/(m³·kg)) of the rotor per the capacity of the emulsifying/dispersing chamber and the amount of the oil dispersion liquid) is preferably 1×10⁴ W·h/(m³·kg) or more, more preferably 1×10⁵ W·h/(m³·kg) or more, and further preferably 0.5×106 W·h/(m³·kg) or more, from the standpoint of enhancing the shearing effect on the oil mixture liquid and producing the fine emulsion liquid having an average particle diameter of 10 μm or less rapidly while suppressing the reaction rate of the silica precursor. The rotation energy Q″ may be preferably 1×10¹² W·h/(m³·kg) or less from the standpoint of the production efficiency.

The treatment flow rate of the in-line emulsifier/disperser is preferably 0.1 L/min or more, more preferably 0.5 L/min or more, further preferably 1 L/min or more, and still further preferably 3 L/min or more, from the standpoint of producing the fine emulsion liquid having an average particle diameter of 10 μm or less rapidly before progress of the reaction of the silica precursor. The treatment flow rate may be preferably 1,000 L/min or less from the standpoint of the easiness in production.

The outermost peripheral speed of the rotor of the in-line emulsifier/disperser is preferably 3 m/s or more, more preferably 5 m/s or more, further preferably 10 m/s or more, still further preferably 15 m/s or more, and still more further preferably 20 m/s or more, from the standpoint of applying desired rotation energy, and is preferably 50 m/s or less, and more preferably 45 m/s or less, from the standpoint of suppressing temperature rise of the liquid in the emulsifying/dispersing chamber.

The number of pass of the in-line emulsifier/disperser in the step 1 in performing the emulsification in the step 1 with the in-line emulsifier/disperser is preferably 1 or more, more preferably 2 or more, and further preferably 3 or more, and the upper limit thereof is not particularly limited, and is preferably 100,000 or less, more preferably 10,000 or less, further preferably 5,000 or less, still further preferably 3,000 or less, still more further preferably 2,000 or less, even further preferably 1,500 or less, and even still further preferably 1,300 or less, in consideration of the production efficiency.

The number of pass shows the number of times of application of shear on the oil mixture liquid with the rotor, and is obtained according to the following calculation expression (III).

$$Number of pass = ( ( emulsification time ( h ) \times 60 ) \times ( circulation flow rate ( kg / min ) ) / ( amount of oil mixture liquid used in step 1 ( kg ) ) \qquad ( III )$$

The liquid temperature of the oil mixture liquid applied to the emulsification in the step 1 is preferably 50° C. or less, more preferably 40° C. or less, further preferably 35° C. or less, and still further preferably 30° C. or less, from the standpoint of suppressing the reaction of the silica precursor. The liquid temperature of the oil mixture liquid applied to the emulsification in the step 1 may be preferably 0° C. or more from the standpoint of the production efficiency.

The emulsification time of the step 1 may be appropriately regulated depending on the production scale and the like, is preferably 12 hours or less, more preferably 10 hours or less, further preferably 8 hours or less, still further preferably 6.5 hours or less, still more further preferably 6 hours or less, even further preferably 5 hours or less, and even still further preferably 4 hours or less, and from the standpoint of the easiness in production, is preferably 0.003 hour or more, more preferably 0.01 hour or more, and further preferably 0. 1 hour or more. By shortening the emulsification time in the step 1, the oil-water contact time can be shortened, and the reaction of the silica precursor can be suppressed.

For example, in the case where the amount of the oil mixture liquid used in the step 1 is 100 kg or more, the emulsification time of the step 1 is preferably 12 hours or less, more preferably 10 hours or less, further preferably 8 hours or less, still further preferably 6.5 hours or less, still more further preferably 6 hours or less, even further preferably 5 hours or less, even still further preferably 4 hours or less, and even still more further preferably 3 hours or less, from the standpoint of suppressing the reaction of the silica precursor, and is preferably 0.1 hour or more from the standpoint of the easiness in production.

The median diameter D₅₀ of the emulsion droplets of the emulsion liquid obtained in the step 1 is preferably 0.1 μm or more, more preferably 0.3 μm or more, further preferably 0.5 μm or more, and still further preferably 0.7 μm or more, from the standpoint of reducing the specific surface area of the resulting silica capsule particles facing the external environment and enhancing the retention of the oil, and is preferably 10 μm or less, more preferably 7 μm or less, further preferably 5 μm or less, still further preferably 3 μm or less, still more further preferably 2 μm or less, and even further preferably 1.5 μm or less, from the standpoint of the dispersion stability of the resulting silica capsule particles.

The median diameter D₅₀ of the emulsion droplets can be measured by the method shown in the examples.

The reaction rate of the silica precursor of the emulsion liquid obtained in the step 1 is preferably 80% or less, more preferably 70% or less, further preferably 60% or less, still further preferably 55% or less, still more further preferably 50% or less, even further preferably 40% or less, even still further preferably 35% or less, and even still more further preferably 30% or less, and is preferably 0% or more, from the standpoint of enhancing the encapsulation rate of the oil, and is preferably 1% or more from the standpoint of the easiness in production.

The reaction rate of the silica precursor can be measured by the method shown in the examples.

### Step 2

### Formation of Silica Capsule Particles

The step 2 is a step of forming oil-containing silica capsule particles by using the emulsion liquid obtained in the step 1, in a batch type agitation tank.

In the step 2, the emulsion liquid is agitated in a batch type agitation tank, and thereby it is considered that the partial or entire breakage of the emulsion droplets formed in the step 1 can be suppressed, the silica capsule particles having a dense and firm shell can be formed, and the encapsulation rate of the oil can be enhanced.

In the step 2, it is preferred that the emulsion liquid obtained in the step 1 is diluted by adding water thereto, and then the oil-containing silica capsule particles are formed in the batch type agitation tank. The diluting operation included can reduce the amount of the oil mixture liquid applied to the emulsification treatment in the step 1, so as to apply the shearing force efficiently to the oil mixture liquid, and thereby the emulsification time can be shortened, the partial or entire breakage of the emulsion droplets formed in the step 1 can be suppressed, and the encapsulation rate of the oil can be enhanced.

The dilution in the step 2 is performed to make the total amount of the oil and the silica precursor used in the step 1 of preferably 35 parts by mass or less, more preferably 30 parts by mass or less, and further preferably 27 parts by mass or less, and from the standpoint of the production efficiency, of preferably 10 parts by mass or more, more preferably 15 parts by mass or more, and further preferably 20 parts by mass or more, per 100 parts by mass of the emulsion liquid after the dilution.

The dilution factor is preferably 1.3 times or more, more preferably 1.4 times or more, and further preferably 1.5 times or more, from the standpoint of enhancing the encapsulation rate of the oil, and is preferably 3 times or less, more preferably 2 times or less, and further preferably 1.8 times or less, from the standpoint of the production efficiency.

In the present invention, the “dilution factor” is the mass ratio of the total amount of the emulsion liquid after the dilution with respect to the total amount of the emulsion liquid before the dilution obtained in the step 1 to be applied to the step 2 ((total amount of emulsion liquid after dilution)/(total amount of emulsion liquid before dilution obtained in step 1 to be applied to step 2)).

The pH of the emulsion liquid to be applied to the formation of silica capsule particles in the step 2 is preferably 3.0 or more, more preferably 3.3 or more, and further preferably 3.5 or more, from the standpoint of retaining the balance between the hydrolysis reaction and the polycondensation reaction of the silica precursor, and the standpoint of suppressing the formation of a highly hydrophilic sol, accelerating the encapsulation of the oil, and enhancing the encapsulation rate of the oil, and is preferably 4.5 or less, more preferably 4.3 or less, and further preferably 4.0 or less, from the standpoint of suppressing the simultaneous occurrence of formation of a silica shell and aggregation of the emulsion droplets, and enhancing the encapsulation rate the oil.

In the step 2, from the standpoint of regulating the emulsion liquid to have a desired pH, corresponding to the pH of the emulsion liquid obtained in the step 1, a pH modifier may be added to the emulsion liquid obtained in the step 1 to regulate the pH of the emulsion liquid, and then the oil-containing silica capsule particles may be formed in the batch type agitation tank.

The pH modifier used may be appropriately selected from an acidic pH modifier and an alkaline pH modifier depending on the pH of the emulsion liquid obtained in the step 1.

Examples of the acidic pH modifier include an inorganic acid, such as hydrochloric acid, nitric acid, and sulfuric acid, an organic acid, such as acetic acid and citric acid, and a liquid obtained by adding a cation exchange resin or the like to water, ethanol, or the like, and one or more kind selected from hydrochloric acid, sulfuric acid, nitric acid, and citric acid is preferred.

Examples of the alkaline pH modifier include sodium hydroxide, sodium hydrogen carbonate, potassium hydroxide, ammonium hydroxide, diethanolamine, triethanolamine, and trishydroxymethylaminomethane, and one or more kind selected from sodium hydroxide and ammonium hydroxide is preferred.

The pH of the emulsion liquid obtained in the step 1 may have pH that is a desired value or less depending on the kind of the oil, and in this case, it is preferred to regulate by using the alkaline pH modifier.

The formation of the oil-containing silica capsule particles in the step 2 is preferably performed while agitating with a disperser using an agitation blade from the standpoint of suppressing the partial or entire breakage of the emulsion droplets formed in the step 1 and enhancing the encapsulation rate of the oil.

Preferred examples of the agitation blade include one or more kind selected from a paddle blade, a turbine blade, an anchor blade, a ribbon blade, and a propeller.

The agitation speed in the step 2 in terms of tip peripheral speed is preferably 200 r/min or less, more preferably 100 r/min or less, and further preferably 90 r/min or less, from the standpoint of suppressing the partial or entire breakage of the emulsion droplets formed in the step 1 and enhancing the encapsulation rate of the oil, and is preferably 10 r/min or more, more preferably 15 r/min or more, and further preferably 20 r/min or more, from the standpoint of providing the silica capsule particles having a narrow particle diameter distribution.

The liquid temperature in the step 2 is preferably 0° C. or more, and is preferably 40° C. or less, and more preferably 35° C. or less.

The agitation time in the step 2 is preferably 6 hours or more, more preferably 12 hours or more, and further preferably 18 hours or more, and is preferably 48 hours or less, more preferably 36 hours or less, and further preferably 30 hours or less, while depending on the production scale, the agitation speed, the temperature condition, and the like.

In the step 2, from the standpoint of enhancing the encapsulation rate of the oil, after forming the oil-containing silica capsule particles using the emulsion liquid obtained in the step 1 (the oil-containing silica capsule particles in this case may be referred to as “oil-containing silica capsule particles (1)”), a silica precursor may be further added to form the oil-containing silica capsule particles. According to the procedure, a second shell encapsulating the first shell of the oil-containing silica capsule particles (1) is formed, and thereby the shell of the resulting oil-containing silica capsule particles can be dense and firm.

The silica precursor used in the case where the silica precursor is further added in the step 2 preferably contains a tetraalkoxysilane, and is more preferably a tetraalkoxysilane having an alkoxy group having 1 or more and 4 or less carbon atoms, further preferably one or more kind selected from tetramethoxysilane, tetraethoxysilane, and tetraisopropoxysilane, still further preferably one or more kind selected from tetramethoxysilane and tetraethoxysilane, and still more further preferably tetraethoxysilane, as similar to the silica precursor described above, from the standpoint of accelerating the sol-gel reaction and enhancing the encapsulation rate of the oil.

In the case where the silica precursor contains a tetraalkoxysilane, the content of the tetraalkoxysilane in the silica precursor is preferably 80% by mass or more, more preferably 85% by mass or more, and further preferably 90% by mass or more, and is preferably 100% by mass or less.

The amount of the silica precursor used in the case where the silica precursor is further added in the step 2 is preferably 5 parts by mass or more, more preferably 7 parts by mass or more, and further preferably 10 parts by mass or more, and is preferably 200 parts by mass or less, more preferably 150 parts by mass or less, and further preferably 100 parts by mass or less, per 100 parts by mass of the oil used in the step 1, from the standpoint of enhancing the encapsulation rate of the oil.

In the case where the silica precursor is further added in the step 2, the preferred ranges of the agitation speed, the liquid temperature, and the agitation time in the formation of the second shell in the step 2 are the same as the preferred ranges of the agitation speed, the liquid temperature, and the agitation time in the formation of the first shell of the oil-containing silica capsule particles (1) described above.

The oil-containing silica capsule particles obtained by the production method of the present invention are preferably obtained in the form of a water dispersion containing the oil-containing silica capsule particles dispersed in water. The water dispersion may be directly used in some applications, and the oil-containing silica capsule particles may be used after separation in some cases. The separation method used may be a filtering method, a centrifugal method, or the like.

The water dispersion of the oil-containing silica capsules obtained by the production method of the present invention may contain an additional component added thereto, such as a pH modifier, a colorant, an antiseptic, an anti-foaming agent, an antioxidant, an ultraviolet ray absorbent, a shell surface modifier, a dispersant, an inorganic salt, a thickener, a deposition aid, and a rheology modifier.

The median diameter D₅₀ of the oil-containing silica capsule particles according to the present invention is preferably 0.1 μm or more, more preferably 0.3 μm or more, further preferably 0.5 μm or more, still further preferably 0.7 μm or more, and still more further preferably 1.0 μm or more, from the standpoint of reducing the specific surface area of the resulting silica capsule particles facing the external environment and enhancing the retention of the oil, and is preferably 10 μm or less, more preferably 7 μm or less, further preferably 5 μm or less, and still further preferably 3 μm or less, from the standpoint of the dispersion stability of the resulting silica capsule particles.

The median diameter D₅₀ of the oil-containing silica capsule particles can be measured by the method shown in the examples.

The oil-containing silica capsule particles according to the present invention can be used in various applications. The oil-containing silica capsule particles according to the present invention can be favorably used, for example, in perfumery and cosmetics, such as an emulsion lotion, a cosmetic lotion, a beauty lotion, a cosmetic cream, a gel formulation, a hair treatment, and a quasi drug; a fiber treatment agent, such as a detergent, a softener, and an anti-wrinkle spray; a sanitary product, such as a disposable diaper; and a perfuming agent.

The oil-containing silica capsule particles according to the present invention can be used by mixing in a composition, such as a detergent composition, a fiber treatment agent composition, a perfumery and cosmetic composition, a perfuming agent composition, and a deodorizer composition. The composition is preferably one or more kind selected from a detergent composition, such as a powder detergent composition and a liquid detergent composition; and a fiber treatment agent composition, such as a softener composition, more preferably a fiber treatment agent composition, and further preferably a softener composition.

In relation to the embodiments described above, the present invention further relates to the following methods for producing oil-containing silica capsule particles.
<1> A method of producing oil-containing silica capsule particles having a core containing an oil and a shell containing silica as a constitutional component, including:
   step 1: a step of emulsifying an oil mixture liquid containing a surfactant, water, an oil, and a silica precursor, with an in-line emulsifier/disperser, so as to provide an emulsion liquid; and
   step 2: a step of forming oil-containing silica capsule particles by using the emulsion liquid obtained in the step 1, in a batch type agitation tank.
<2> The production method according to the item <1>, wherein the emulsion liquid obtained in the step 1 has a reaction rate of the silica precursor of 0% or more and 80% or less.

<3> The production method according to the item <1> or <2>, wherein the emulsion liquid obtained in the step 1 has a reaction rate of the silica precursor of 0% or more and 60% or less.
<4> The production method according to any one of the items <1> to <3>, wherein the emulsion liquid obtained in the step 1 has a reaction rate of the silica precursor of 0% or more and 55% or less.
<5> The production method according to any one of the items <1> to <4>, wherein the emulsion liquid obtained in the step 1 has a reaction rate of the silica precursor of 0% or more and 50% or less.
<6> The production method according to any one of the items <1> to <5>, wherein the in-line emulsifier/disperser used in the step 1 has a rotor and a stator.
<7> The production method according to any one of the items <1> to <6>, wherein the in-line emulsifier/disperser used in the step 1 is Cavitron or Milder.
<8> The production method according to any one of the items <1> to <7>, wherein in the emulsification in the step 1, the rotation energy Q of the rotor per the capacity of the emulsifying/dispersing chamber of the in-line emulsifier/disperser is 1×10⁷ W/m³ or more and 1×10¹² W/m³ or less.
<9> The production method according to any one of the items <1> to <8>, wherein in the emulsification in the step 1, the rotation energy Q′ of the rotor per the capacity of the emulsifying/dispersing chamber of the in-line emulsifier/disperser is 1×10⁶ W·h/m³ or more and 1×10¹² W·h/m³ or less.
<10> The production method according to any one of the items <1> to <9>, wherein in the emulsification in the step 1, the rotation energy Q″ of the rotor per the capacity of the emulsifying/dispersing chamber of the in-line emulsifier/disperser and the amount of the oil dispersion liquid is 1×10⁴ W·h/(m³·kg) or more and 1×10¹² W·h/(m³·kg) or less.
<11> The production method according to any one of the items <1> to <10>, wherein in the emulsification in the step 1, the treatment flow rate of the in-line emulsifier/disperser is 0.1 L/min or more and 1,000 L/min or less.
<12> The production method according to any one of the items <1> to <11>, wherein the outermost peripheral speed of the rotor of the in-line emulsifier/disperser is 3 m/s or more and 50 m/s or less.
<13> The production method according to any one of the items <1> to <12>, wherein the outermost peripheral speed of the rotor of the in-line emulsifier/disperser is 5 m/s or more and 45 m/s or less.
<14> The production method according to any one of the items <1> to <13>, wherein the amount of the oil mixture liquid used in the step 1 is 100 kg or more.
<15> The production method according to any one of the items <1> to <14>, wherein the amount of the oil mixture liquid used in the step 1 is 300 kg or more and 100,000 kg or less.
<16> The production method according to any one of the items <1> to <15>, wherein the amount of the oil mixture liquid used in the step 1 is 500 kg or more and 100,000 kg or less.
<17> The production method according to any one of the items <1> to <16>, wherein the amount of the oil mixture liquid used in the step 1 is 1,000 kg or more and 100,000 kg or less.
<18> The production method according to any one of the items <1> to <17>, wherein the emulsification time of the step 1 is 12 hours or less.
<19> The production method according to any one of the items <1> to <18>, wherein the emulsification time of the step 1 is 0.1 hour or more and 10 hours or less.
<20> The production method according to any one of the items <1> to <19>, wherein the emulsification time of the step 1 is 0.1 hour or more and 8 hours or less.
<21> The production method according to any one of the items <1> to <20>. wherein the emulsification time of the step 1 is 0.1 hour or more and 6.5 hours or less.
<22> The production method according to any one of the items <1> to <21>, wherein the emulsification time of the step 1 is 0.1 hour or more and 6 hours or less.
<23> The production method according to any one of the items <1> to <22>, wherein the oil mixture liquid applied to the emulsification in the step 1 has a liquid temperature of 0° C. or more and 50° C. or less.
<24> The production method according to any one of the items <1> to <23>, wherein the oil mixture liquid applied to the emulsification in the step 1 has a liquid temperature of 0° C. or more and 40° C. or less.
<25> The production method according to any one of the items <1> to <24>, wherein the oil mixture liquid applied to the emulsification in the step 1 has a liquid temperature of 0° C. or more and 35° C. or less.
<26> The method of producing oil-containing silica capsule particles according to any one of the items <1> to <25>, wherein the silica precursor contains a tetraalkoxysilane,
<27> The production method according to any one of the items <1> to <26>, wherein the silica precursor is a tetraalkoxysilane having an alkoxy group having 1 or more and 4 or less carbon atoms.
<28> The production method according to any one of the items <1> to <27>, wherein the silica precursor is one or more kind selected from tetramethoxysilane, tetraethoxysilane, and tetraisopropoxysilane.
<29> The production method according to any one of the items <1> to <28>, wherein in the step 2, a pH modifier is added to the emulsion liquid obtained in the step 1 to regulate the pH of the emulsion liquid to 4.5 or less, and then the oil-containing silica capsule particles are formed in the batch type agitation tank.
<30> The production method according to the item <29>, wherein the pH of the emulsion liquid is 3.0 or more and 4.5 or less.
<31> The production method according to the item <29> or <30>, wherein the pH of the emulsion liquid is 3.3 or more and 4.3 or less.
<32> The production method according to any one of the items <1> to <31>, wherein in the step 2, the emulsion liquid obtained in the step 1 is diluted by adding water thereto, and then the oil-containing silica capsule particles are formed in the batch type agitation tank.
<33> The production method according to the item <32>, wherein the dilution in the step 2 is performed to make the total amount of the oil and the silica precursor used in the step 1 of 35 parts by mass or less per 100 parts by mass of the emulsion liquid after the dilution.
<34> The production method according to any one of the items <1> to <33>, wherein the mass ratio of the water phase component containing the surfactant and water and the oil phase component containing the oil and the silica precursor of the oil mixture liquid used in the step 1 (water phase component/oil phase component) is 50/50 or more and 99/1 or less.
<35> The production method according to any one of the items <1> to <34>, wherein the mass ratio of the water phase component containing the surfactant and water and the oil phase component containing the oil and the silica precursor of the oil mixture liquid used in the step 1 (water phase component/oil phase component) is 53/47 or more and 90/10 or less.
<36> The production method according to any one of the items <1> to <35>, wherein the emulsification in the step 1 is performed by using the in-line emulsifier/disperser by an external circulation system.
<37> The production method according to any one of the items <1> to <35>, wherein the emulsification in the step 1 is performed in such a manner that the surfactant, water, the oil, and the silica precursor are mixed in in-line mode to prepare the oil mixture liquid, and then the oil mixture liquid is supplied to the in-line emulsifier/disperser.
<38> The production method according to the item <36> or <37>, wherein the number of pass of the in-line emulsifier/disperser in the step 1 is 1 or more and 100,000 or less.
<39> The production method according to any one of the items <36> to <38>, wherein the number of pass of the in-line emulsifier/disperser in the step 1 is 1 or more and 10,000 or less.
<40> The production method according to any one of the items <36> to <39>, wherein the number of pass of the in-line emulsifier/disperser in the step 1 is 2 or more and 5,000 or less.
<41> The production method according to any one of the items <36> to <40>, wherein the number of pass of the in-line emulsifier/disperser in the step 1 is 3 or more and 3,000 or less.
<42> The production method according to any one of the items <1> to <41>, wherein the emulsion droplets of the emulsion liquid obtained in the step 1 have a median diameter D₅₀ of 10 μm or less.
<43> The production method according to any one of the items <1> to <42>, wherein the emulsion droplets of the emulsion liquid obtained in the step 1 have a median diameter D₅₀ of 0.1 μm or more and 7 μm or less.
<44> The production method according to any one of the items <1> to <43>, wherein the emulsion droplets of the emulsion liquid obtained in the step 1 have a median diameter D₅₀ of 0.3 μm or more and 5 μm or less.
<45> The production method according to any one of the items <1> to <44>, wherein the emulsion droplets of the emulsion liquid obtained in the step 1 have a median diameter D₅₀ of 0.5 μm or more and 3 μm or less.
<46> The production method according to any one of the items <1> to <45>, wherein in the step 2, a disperser using an agitation blade is used.
<47> The production method according to the item <46>, wherein the agitation blade is one or more kind selected from a paddle blade, a turbine blade, an anchor blade, a ribbon blade, and a propeller.
<48> The production method according to the item <46> or <47>, wherein the agitation speed in the step 2 is 10 r/min or more and 200 r/min or less.
<49> The production method according to any one of the items <46> to <48>, wherein the agitation speed in the step 2 is 15 r/min or more and 100 r/min or less.
<50> The production method according to any one of the items <46> to <49>, wherein the agitation speed in the step 2 is 20 r/min or more and 90 r/min or less.

### EXAMPLES

The measurements and calculations used in the examples were performed by the following methods.

### Median Diameter D₅₀

The median diameter D₅₀ of the emulsion droplets and the median diameter D₅₀ of the oil-containing silica capsule particles were measured by using a laser diffraction/scattering particle diameter distribution measuring device, “LA-960” (trade name, available from Horiba, Ltd.). In the measurement, a flow cell was used, water was used as a medium, and the refractive index of the dispersoid was set to 1.45-0i. The emulsion liquid or the water dispersion containing the oil-containing silica capsule particles was added to the flow cell, and measured at a concentration showing a transmittance of approximately 90%, so as to obtain the volume based median diameter D₅₀.

### Reaction Rate of Silica Precursor

100 mg of the oil mixture liquid to be applied to the emulsification of the step 1 was diluted with 10 g of methanol containing dodecane as an internal standard in a concentration of 10 μg/mL, and then the diluted liquid was measured by gas chromatography, so as to measure the amount β of the silica precursor in 100 mg of the oil mixture liquid.

Subsequently, 100 mg of the emulsion liquid obtained in the step 1 was diluted with 10 g of methanol containing dodecane as an internal standard in a concentration of 10 μg/mL, and then the diluted liquid was measured by gas chromatography, so as to measure the amount a of the unreacted silica precursor in 100 mg of the emulsion liquid. The reaction rate of the silica precursor was calculated according to the following expression.

Reaction rate of silica precursor (%)=100−((amount a of unreacted silica precursor contained in 100 mg of emulsion liquid)/(amount β of silica precursor contained in 100 mg of oil mixture liquid)×100

### <Model Perfume>

As the oil encapsulated in the silica capsule particles, a model perfume A (volume average cLogP: 3.7, specific gravity: 0.96) having the composition shown in Table 1 was used. The volume average cLogP value of the model perfume was calculated in such a manner that the cLogP values of the perfume components contained in the model perfume were multiplied by the volume proportions thereof respectively, and the resulting values were summated.

TABLE 1
Model Perfume A

| Perfume component | Content (% by mass) | cLogP |
|---|---|---|
| Methyl dihydrojasmonate | 20.6 | 3.0 |
| γ-Decalactone | 13.5 | 2.6 |
| Ethylene brassylate | 12.0 | 4.7 |
| o-t-Butylcyclohexyl acetate | 11.0 | 4.4 |
| Amber core | 8.0 | 4.1 |
| Others | 34.9 | |

### Example 1

### (Step 1)

In a spherical bottom cylindrical agitation tank having an inner capacity of 300 L (inner diameter: 0.70 m) equipped with a 45°-inclined paddle blade (blade diameter: 0.35 m), 0.7 kg of Quartamin 60W (trade name, cetyltrimethylammonium chloride, active ingredient: 30% by mass, available from Kao Corporation) and 89.4 kg of ion exchanged water were mixed at a temperature of 15° C. and a rotation number in agitation of 80 r/min for 10 minutes to prepare a water phase component. 47.9 kg of the model perfume A as an oil and 12.0 kg of tetraethoxysilane (which may be hereinafter referred to as “TEOS”) as a silica precursor were preliminarily mixed in a drum can having an inner capacity of 200 L to prepare an oil phase component, which was added to the water phase component to provide an oil mixture liquid.

150.0 kg of the resulting oil mixture liquid was emulsified at a temperature of 15° C. with the paddle blade at a rotation number in agitation of 80 r/min by circulating by the external circulation system for 20 minutes with an in-line emulsifier/disperser (Cavitron CD1010, trade name, available from Eurotec Co., Ltd.) set to an outermost peripheral speed of the rotor of 40 m/s, while circulating and mixing at a flow rate of 40 L/min with an air-driven diaphragm pump, so as to provide an emulsion liquid. The median diameter D₅₀ of the emulsion droplets of the resulting emulsion liquid and the reaction rate of the silica precursor are shown in Table 2.

### (Step 2)

The emulsion liquid obtained in the step 1 was diluted by adding 89.4 kg of ion exchanged water thereto, to which a 1% by mass sulfuric acid aqueous solution as a pH modifier was added to regulate the pH to 3.7, and then agitated with the paddle blade at 80 r/min for 24 hours in the agitation tank while increasing and retaining the liquid temperature to 30° C., so as to provide a water dispersion containing oil-containing silica capsule particles shown in Table 2.

In Example 1, the period of time required for producing 100 kg of the emulsion liquid as an index of the emulsion productivity was 0.2 hour. A shorter period thereof means better emulsion productivity.

### Example 2

### (Step 1)

In a spherical bottom cylindrical agitation tank having an inner capacity of 6.5 m³ (inner diameter: 1.9 m) equipped with a 45°-inclined paddle blade (blade diameter: 1.3 m), 6.9 kg of Quartamin 60W and 879.1 kg of ion exchanged water were mixed at a temperature of 15° C. and a rotation number in agitation of 35 r/min for 10 minutes to prepare a water phase component. 471.0 kg of the model perfume A as an oil and 118.0 kg of tetraethoxysilane as a silica precursor were preliminarily mixed in a spherical bottom cylindrical tank having an inner capacity of 6.5 m³ to prepare an oil phase component, which was added to the water phase component to provide an oil mixture liquid.

1,475.0 kg of the resulting oil mixture liquid was emulsified at a temperature of 15° C. with the paddle blade at a rotation number in agitation of 35 r/min by circulating by the external circulation system for 150 minutes with an in-line emulsifier/disperser (Cavitron CD1010, trade name, available from Eurotec Co., Ltd.) set to an outermost peripheral speed of the rotor of 40 m/s, while circulating and mixing at a flow rate of 50 L/min with an air-driven diaphragm pump, so as to provide an emulsion liquid. The median diameter D₅₀ of the emulsion droplets of the resulting emulsion liquid and the reaction rate of the silica precursor are shown in Table 2.

### (Step 2)

The emulsion liquid obtained in the step 1 was diluted by adding 879.1 kg of ion exchanged water thereto, to which a 1% by mass sulfuric acid aqueous solution as a pH modifier was added to regulate the pH to 3.7, and then agitated with the paddle blade at 35 r/min for 24 hours in the agitation tank while increasing and retaining the liquid temperature to 30° C., so as to provide a water dispersion containing oil-containing silica capsule particles shown in Table 2.

In Example 2, the period of time required for producing 100 kg of the emulsion liquid as an index of the emulsion productivity was 0.2 hour.

### Example 3

### (Step 1)

In a spherical bottom cylindrical agitation tank having an inner capacity of 15 m³ (inner diameter: 2.5 m) equipped with a 45°-inclined paddle blade (blade diameter: 1.9 m), 42.2 kg of Quartamin 60W and 5,384.3 kg of ion exchanged water were mixed at a temperature of 15° C. and a rotation number in agitation of 27 r/min for 10 minutes to prepare a water phase component. 2,884.9 kg of the model perfume A as an oil and 722.7 kg of tetraethoxysilane as a silica precursor were preliminarily mixed in a spherical bottom cylindrical tank having an inner capacity of 15 m³ to prepare an oil phase component, which was added to the water phase component to provide an oil mixture liquid.

9,034.0 kg of the resulting oil mixture liquid was emulsified at a temperature of 15° C. with the paddle blade at a rotation number in agitation of 27 r/min by circulating by the external circulation system for 140 minutes with an in-line emulsifier/disperser (Cavitron CD1030, trade name, available from Eurotec Co., Ltd.) set to an outermost peripheral speed of the rotor of 23 m/s, while circulating and mixing at a flow rate of 300 L/min with an air-driven diaphragm pump, so as to provide an emulsion liquid. The median diameter D₅₀ of the emulsion droplets of the resulting emulsion liquid and the reaction rate of the silica precursor are shown in Table 2.

### (Step 2)

The emulsion liquid obtained in the step 1 was diluted by adding 5,384.3 kg of ion exchanged water thereto, to which a 1% by mass sulfuric acid aqueous solution as a pH modifier was added to regulate the pH to 3.7, and then agitated with the paddle blade at 27 r/min for 24 hours in the agitation tank while increasing and retaining the liquid temperature to 30° C., so as to provide a water dispersion containing oil-containing silica capsule particles shown in Table 2.

In Example 3, the period of time required for producing 100 kg of the emulsion liquid as an index of the emulsion productivity was 0.03 hour.

### Example 4

### (Step 1)

In a spherical bottom cylindrical agitation tank having an inner capacity of 5 L equipped with a 45°-inclined paddle blade (blade diameter: 0.35 m), 9.3 g of Quartamin 60W and 1,192.0 g of ion exchanged water were mixed at a temperature of 15° C. and a rotation number in agitation of 80 r/min for 10 minutes to prepare a water phase component. 638.7 g of the model perfume A as an oil and 160.0 g of tetraethoxysilane as a silica precursor were preliminarily mixed in a resin container having an inner capacity of 2 L to prepare an oil phase component, which was added to the water phase component to provide an oil mixture liquid.

2 kg of the resulting oil mixture liquid was emulsified at a temperature of 15° C. with the paddle blade at a rotation number in agitation of 27 r/min by circulating by the external circulation system for 3 hours and 59 minutes with an in-line emulsifier/disperser (Cavitron CD1000, trade name, available from Eurotec Co., Ltd.) set to an outermost peripheral speed of the rotor of 5 m/s, while circulating and mixing at a flow rate of 7 L/min with an air-driven diaphragm pump, and subsequently further emulsified by circulating for 1 minute at an outermost peripheral speed of the rotor of 23 m/s, so as to provide an emulsion liquid. The median diameter D₅₀ of the emulsion droplets of the resulting emulsion liquid and the reaction rate of the silica precursor are shown in Table 2.

### (Step 2)

The emulsion liquid obtained in the step 1 was diluted by adding 1,192.0 g of ion exchanged water thereto, to which a 1% by mass sulfuric acid aqueous solution as a pH modifier was added to regulate the pH to 3.7, and then agitated with the paddle blade at 27 r/min for 24 hours in the agitation tank while increasing and retaining the liquid temperature to 30° C., so as to provide a water dispersion containing oil-containing silica capsule particles shown in Table 2.

### Example 5

A water dispersion containing oil-containing silica capsule particles shown in Table 2 was obtained in the same manner as in Example 4 except that in the step 1 of Example 4, the oil mixture liquid was emulsified by circulating by the external circulation system for 5 hours and 59 minutes with an in-line emulsifier/disperser (Cavitron CD1000, trade name, available from Eurotec Co., Ltd.) set to an outermost peripheral speed of the rotor of 5 m/s, and then emulsified by circulating for 1 minute at an outermost peripheral speed of the rotor of 23 m/s.

### Example 6

A water dispersion containing oil-containing silica capsule particles shown in Table 2 was obtained in the same manner as in Example 4 except that in the step 1 of Example 4, the oil mixture liquid was emulsified at a liquid temperature of 30° C.

### Example 7

A water dispersion containing oil-containing silica capsule particles shown in Table 2 was obtained in the same manner as in Example 5 except that in the step 1 of Example 5, the oil mixture liquid was emulsified at a liquid temperature of 30° C.

### Example 8

A water dispersion containing oil-containing silica capsule particles was obtained in the same manner as in Example 5 except that in the step 1 of Example 5, the oil mixture liquid was emulsified by circulating by the external circulation system for 9 hours and 59 minutes with an in-line emulsifier/disperser (Cavitron CD1000, trade name, available from Eurotec Co., Ltd.) set to an outermost peripheral speed of the rotor of 5 m/s, and then emulsified by circulating for 1 minute at an outermost peripheral speed of the rotor of 23 m/s.

### Example 9

A water dispersion containing oil-containing silica capsule particles shown in Table 2 was obtained in the same manner as in Example 7 except that in the step 1 of Example 7, the oil mixture liquid was emulsified by circulating by the external circulation system for 6 hours and 59 minutes with an in-line emulsifier/disperser (Cavitron CD1000, trade name, available from Eurotec Co., Ltd.) set to an outermost peripheral speed of the rotor of 5 m/s, and then emulsified by circulating for 1 minute at an outermost peripheral speed of the rotor of 23 m/s.

### Example 10

A water dispersion containing oil-containing silica capsule particles shown in Table 2 was obtained in the same manner as in Example 4 except that in the step 1 of Example 4, 2 kg of the oil mixture liquid was preliminarily emulsified by agitating at a temperature of 15° C. with a 45°-inclined paddle blade (blade diameter: 0.35 m) at a rotation number in agitation of 27 r/min for 10 minutes, and then the resulting preliminary emulsion liquid was emulsified by the one-pass system of allowing to pass only once through an in-line emulsifier/disperser (Cavitron CD1000, trade name, available from Eurotec Co., Ltd.) at an outermost peripheral speed of the rotor of 40 m/s.

### Example 11

A water dispersion containing oil-containing silica capsule particles shown in Table 2 was obtained in the same manner as in Example 1 except that in the step 1 of Example 1, 150.0 kg of the oil mixture liquid was preliminarily emulsified by agitating at a temperature of 15° C. with a 45°-inclined paddle blade (blade diameter: 0.35 m) at a rotation number in agitation of 80 r/min for 10 minutes, and then the resulting preliminary emulsion liquid was emulsified by the one-pass system of allowing to pass only once through an in-line emulsifier/disperser (Cavitron CD1010, trade name, available from Eurotec Co., Ltd.) at an outermost peripheral speed of the rotor of 40 m/s.

### Example 12

A water dispersion containing oil-containing silica capsule particles shown in Table 2 was obtained in the same manner as in Example 3 except that in the step 1 of Example 3, 9,034.0 kg of the oil mixture liquid was preliminarily emulsified by agitating at a temperature of 15° C. with a 45°-inclined paddle blade (blade diameter: 1.9 m) at a rotation number in agitation of 27 r/min for 10 minutes, and then the resulting preliminary emulsion liquid was emulsified by the one-pass system of allowing to pass only once through an in-line emulsifier/disperser (Cavitron CD1030, trade name, available from Eurotec Co., Ltd.) at an outermost peripheral speed of the rotor of 40 m/s.

### Example 13

A water dispersion containing oil-containing silica capsule particles shown in Table 2 was obtained in the same manner as in Example 4 except that in the step 1 of Example 4, 2 kg of the oil mixture liquid was preliminarily emulsified by agitating at a temperature of 15° C. with a 45°-inclined paddle blade (blade diameter: 0.35 m) at a rotation number in agitation of 27 r/min for 10 minutes, and then with the temperature and the rotation number in agitation in the preliminary emulsification retained, emulsified by circulating by the external circulation system for 1.5 minutes with an in-line emulsifier/disperser (Cavitron CD1000, trade name, available from Eurotec Co., Ltd.) set to an outermost peripheral speed of the rotor of 40 m/s, while circulating and mixing at a flow rate of 7 L/min with an air-driven diaphragm pump.

### Example 14

A water dispersion containing oil-containing silica capsule particles shown in Table 2 was obtained in the same manner as in Example 4 except that in the step 1 of Example 4, 2 kg of the oil mixture liquid was preliminarily emulsified by agitating at a temperature of 15° C. with a 45°-inclined paddle blade (blade diameter: 0.35 m) at a rotation number in agitation of 27 r/min for 10 minutes, and then with the temperature and the rotation number in agitation in the preliminary emulsification retained, emulsified by circulating by the external circulation system for 4 minutes with an in-line emulsifier/disperser (Cavitron CD1000, trade name, available from Eurotec Co., Ltd.) set to an outermost peripheral speed of the rotor of 40 m/s, while circulating and mixing at a flow rate of 7 L/min with an air-driven diaphragm pump.

### Example 15

A water dispersion containing oil-containing silica capsule particles shown in Table 2 was obtained in the same manner as in Example 4 except that in the step 1 of Example 4, 2 kg of the oil mixture liquid was preliminarily emulsified by agitating at a temperature of 15° C. with a 45°-inclined paddle blade (blade diameter: 0.35 m) at a rotation number in agitation of 27 r/min for 10 minutes, and then with the temperature and the rotation number in agitation in the preliminary emulsification retained, emulsified by circulating by the external circulation system for 12.5 minutes with an in-line emulsifier/disperser (Cavitron CD1000, trade name, available from Eurotec Co., Ltd.) set to an outermost peripheral speed of the rotor of 40 m/s, while circulating and mixing at a flow rate of 7 L/min with an air-driven diaphragm pump.

The encapsulation rate of the oil encapsulated in the oil-containing silica capsule particles obtained in each of Examples was calculated and evaluated in the following manner. The results are shown in Table 2.

### Encapsulation Rate of Oil

100 mg of the water dispersion containing the oil-containing silica capsule particles obtained in the step 2 was diluted with 10 g of methanol containing dodecane as an internal standard in a concentration of 10 μg/mL, and then irradiated with an ultrasonic wave by using an ultrasonic wave irradiation device (Model 5510, available from Branson Ultrasonics Corporation) under conditions of an output power of 180 W and an oscillating frequency of 42 kHz for 60 minutes, so as to provide a diluted liquid having the oil eluted from the silica capsule particles. Subsequently, methyl dihydrojasmonate contained in the diluted liquid was measured by gas chromatography, and the amount y of methyl dihydrojasmonate in 100 mg of the water dispersion containing the oil-containing silica capsule particles was measured as the mixed amount of the oil.

Separately, 100 mg of the water dispersion containing the oil-containing silica capsule particles obtained in the step 2 was diluted with 10 g of ion exchanged water, and then allowed to pass through a membrane filter (Omnipore, Model JAWP04700, available from Millipore Corporation), so as to recover the oil-containing silica capsule particles on the membrane filter.

Furthermore, the oil-containing silica capsule particles were rinsed with 10 mL of ion exchanged water and then 10 mL of hexane on the membrane filter, and then the silica capsule particles were immersed in 2 mL of acetonitrile containing dodecane as an internal standard in a concentration of 10 μg/mL, and irradiated with an ultrasonic wave by using an ultrasonic wave irradiation device (Model 5510, available from Branson Ultrasonics Corporation) under conditions of an output power of 180 W and an oscillating frequency of 42 kHz for 60 minutes, so as to elute the oil from the silica capsule particles. After allowing the solution to pass again through a membrane filter (Dismic, Model 13JP020AN, available from Toyo Roshi Kaisha, Ltd.), methyl dihydrojasmonate contained in the solution was measured by gas chromatography, and the amount x of methyl dihydrojasmonate encapsulated in the silica capsule particles was obtained. The encapsulation rate of the oil was calculated according to the following expression.

Encapsulation rate of oil (%)=((amount x of methyl dihydrojasmonate encapsulated in oil-containing silica capsule particles in 100 mg of water dispersion containing oil-containing silica capsule particles)/(amount y of methyl dihydrojasmonate in 100 mg of water dispersion containing oil-containing silica capsule particles))×100

TABLE 2

| | | | | Example | | | | | | | |
|---|---|---|---|---|---|---|---|---|---|---|---|
| | | | | 1 | 2 | 3 | 4 | 5 | 6 | 7 | 8 |
| Step 1 | Prepared | Water phase | Quartamin | 0.7 kg | 6.9 kg | 42.2 kg | 9.3 g | 9.3 g | 9.3 g | 9.3 g | 9.3 g |
| | composition | component | 60 W *1 | | | | | | | | |
| | of oil | | Ion exchanged | 89.4 kg | 879.1 kg | 5384.3 kg | 1192.0 g | 1192.0 g | 1192.0 g | 1192.0 g | 1192.0 g |
| | mixture | | water | | | | | | | | |
| | liquid | Oil phase | Model | 47.9 kg | 471.0 kg | 2884.9 kg | 638.7 g | 638.7 g | 638.7 g | 638.7 g | 638.7 g |
| | | component | perfume A | | | | | | | | |
| | | | TEOS | 12.0 kg | 118.0 kg | 722.7 kg | 160.0 g | 160.0 g | 160.0 g | 160.0 g | 160.0 g |

| | | | | | | | | | |
|---|---|---|---|---|---|---|---|---|---|
| | Mass ratio (water phase component/ | 60/40 | 60/40 | 60/40 | 60/40 | 60/40 | 60/40 | 60/40 | 60/40 |
| | oil phase component) | | | | | | | | |
| | Amount of oil mixture liquid (kg) | 150 | 1475 | 9034 | 2 | 2 | 2 | 2 | 2 |
| | Liquid temperature of oil mixture | 15 | 15 | 15 | 15 | 15 | 30 | 30 | 15 |
| | liquid (° C.) | | | | | | | | |
| | Kind of emulsifier/disperser | Cavitron | Cavitron | Cavitron | Cavitron | Cavitron | Cavitron | Cavitron | Cavitron |
| | | CD1010 | CD1010 | CD1030 | CD1000 | CD1000 | CD1000 | CD1000 | CD1000 |
| | Outermost peripheral speed of rotor | 40 | 40 | 23 | 5 => 23 | 5 => 23 | 5 => 23 | 5 => 23 | 5 => 23 |
| | (m/s) | | | | | | | | |
| | n: Rotation number of rotor | 187 | 187 | 46 | 150 | 150 | 150 | 150 | 150 |
| | (s⁻¹) | | | | | | | | |
| | d: Outer diameter of rotor (m) | 0.0683 | 0.0683 | 0.16 | 0.0485 | 0.0485 | 0.0485 | 0.0485 | 0.0485 |
| | ρ: Density of oil mixture liquid | 1000 | 1000 | 1000 | 1000 | 1000 | 1000 | 1000 | 1000 |
| | (kg/m³) | | | | | | | | |
| | Capacity of emulsifying/dispersing | 3.32 × | 3.32 × | 3.28 × | 2.34 × | 2.34 × | 2.34 × | 2.34 × | 2.34 × |
| | chamber of in-line emulsifier/ | 10⁻⁷ | 10⁻⁷ | 10⁻⁶ | 10⁻⁷ | 10⁻⁷ | 10⁻⁷ | 10⁻⁷ | 10⁻⁷ |
| | disperser (m³) | | | | | | | | |
| | Emulsification time | 0.3 | 2.5 | 2.3 | 4 | 6 | 4 | 6 | 10 |
| | (oil-water contact time) (h) | | | | | | | | |
| | Rotation energy Q | 2.9 × | 2.9 × | 3.1 × | 3.8 × | 3.8 × | 3.8 × | 3.8 × | 3.8 × |
| | (W/m³) | 10¹⁰ | 10¹⁰ | 10⁹ | 10⁹ | 10⁹ | 10⁹ | 10⁹ | 10⁹ |
| | Rotation energy Q′ | 8.7 × | 7.3 × | 7.3 × | 1.5 × | 2.3 × | 1.5 × | 2.3 × | 3.8 × |
| | (W · h/m³) | 10⁹ | 10¹⁰ | 10⁹ | 10¹⁰ | 10¹⁰ | 10¹⁰ | 10¹⁰ | 10¹⁰ |
| | Rotation energy Q″ | 5.8 × | 4.9 × | 0.8 × | 0.8 × | 1.1 × | 0.8 × | 1.1 × | 1.9 × |
| | (W · h/(m³ · kg)) *2 | 10⁷ | 10⁷ | 10⁶ | 10¹⁰ | 10¹⁰ | 10¹⁰ | 10¹⁰ | 10¹⁰ |
| | Treatment flow rate (L/min) | 40 | 50 | 300 | 7 | 7 | 7 | 7 | 7 |
| | Number of pass | 4.8 | 5.1 | 4.6 | 840 | 1260 | 840 | 1260 | 2100 |
| | Median diameter D₅₀ of | 1.0 | 1.0 | 0.9 | 1.0 | 1.0 | 1.0 | 1.0 | 1.0 |
| | emulsion droplets (μm) | | | | | | | | |
| | Reaction rate of silica precursor (%) | 5 | 15 | 30 | 21 | 37 | 30 | 47 | 52 |
| Step 2 | Amount of ion exchanged water added | 89.4 kg | 879.1 kg | 5384.3 kg | 1192.0 g | 1192.0 g | 1192.0 g | 1192.0 g | 1192.0 g |
| | Total amount of oil and silica precursor | 25 | 25 | 25 | 25 | 25 | 25 | 25 | 25 |
| | used in step 1 per 100 parts by mass of | | | | | | | | |
| | total amount of diluted emulsion liquid | | | | | | | | |
| | (part by mass) | | | | | | | | |
| | Dilution factor (time) | 1.6 | 1.6 | 1.6 | 1.6 | 1.6 | 1.6 | 1.6 | 1.6 |
| | pH of emulsion liquid after regulating pH | 3.7 | 3.7 | 3.7 | 3.7 | 3.7 | 3.7 | 3.7 | 3.7 |
| | Agitation blade | paddle | paddle | paddle | paddle | paddle | paddle | paddle | paddle |
| | | blade | blade | blade | blade | blade | blade | blade | blade |
| | Agitation speed (r/min) | 80 | 35 | 27 | 27 | 27 | 27 | 27 | 27 |

| | | | | | | | | | |
|---|---|---|---|---|---|---|---|---|---|
| Oil-containing silica | Median diameter | 2.0 | 2.0 | 2.0 | 2.0 | 2.0 | 2.0 | 2.0 | 2.0 |
| capsule particles | D₅₀ (μm) | | | | | | | | |
| | Encapsulation | 95 | 95 | 99 | 100 | 98 | 97 | 95 | 48 |
| | rate of oil (%) | | | | | | | | |

| | | | | Example | | | | | | |
|---|---|---|---|---|---|---|---|---|---|---|
| | | | | 9 | 10 | 11 | 12 | 13 | 14 | 15 |
| Step 1 | Prepared | Water phase | Quartamin | 9.3 g | 9.3 g | 0.7 kg | 42.2 kg | 9.3 g | 9.3 g | 9.3 g |
| | composition | component | 60 W *1 | | | | | | | |
| | of oil | | Ion exchanged | 1192.0 g | 1192.0 g | 89.4 kg | 5384.3 kg | 1192.0 g | 1192.0 g | 1192.0 g |
| | mixture | | water | | | | | | | |
| | liquid | Oil phase | Model | 638.7 g | 638.7 g | 47.9 kg | 2884.9 kg | 638.7 g | 638.7 g | 570.5 g |
| | | component | perfume A | | | | | | | |
| | | | TEOS | 160.0 g | 160.0 g | 12.0 kg | 722.7 kg | 160.0 g | 160.0 g | 228.2 g |

| | | | | | | | | |
|---|---|---|---|---|---|---|---|---|
| | Mass ratio (water phase component/ | 60/40 | 60/40 | 60/40 | 60/40 | 60/40 | 60/40 | 60/40 |
| | oil phase component) | | | | | | | |
| | Amount of oil mixture liquid (kg) | 2 | 2 | 150 | 9034 | 2 | 2 | 2 |
| | Liquid temperature of oil mixture | 30 | 15 | 15 | 15 | 15 | 15 | 15 |
| | liquid (° C.) | | | | | | | |
| | Kind of emulsifier/disperser | Cavitron | Cavitron | Cavitron | Cavitron | Cavitron | Cavitron | Cavitron |
| | | CD1000 | CD1000 | CD1010 | CD1030 | CD1000 | CD1000 | CD1000 |
| | Outermost peripheral speed of rotor | 5 => 23 | 40 | 40 | 40 | 40 | 40 | 40 |
| | (m/s) | | | | | | | |
| | n: Rotation number of rotor | 150 | 260 | 187 | 80 | 260 | 260 | 260 |
| | (s⁻¹) | | | | | | | |
| | d: Outer diameter of rotor (m) | 0.0485 | 0.0485 | 0.0683 | 0.16 | 0.0485 | 0.0485 | 0.0485 |
| | ρ: Density of oil mixture liquid | 1000 | 1000 | 1000 | 1000 | 1000 | 1000 | 1000 |
| | (kg/m³) | | | | | | | |
| | Capacity of emulsifying/dispersing | 2.34 × | 2.34 × | 3.32 × | 3.28 × | 2.34 × | 2.34 × | 2.34 × |
| | chamber of in-line emulsifier/ | 10⁻⁷ | 10⁻⁷ | 10⁻⁷ | 10⁻⁶ | 10⁻⁷ | 10⁻⁷ | 10⁻⁷ |
| | disperser (m³) | | | | | | | |
| | Emulsification time | 7 | 0.004 | 0.1 | 0.5 | 0.02 | 0.06 | 0.2 |
| | (oil-water contact time) (h) | | | | | | | |
| | Rotation energy Q | 3.8 × | 2.0 × | 2.9 × | 1.6 × | 2.0 × | 2.0 × | 2.0 × |
| | (W/m³) | 10⁹ | 10¹⁰ | 10¹⁰ | 10¹⁰ | 10¹⁰ | 10¹⁰ | 10¹⁰ |
| | Rotation energy Q′ | 2.7 × | 8.1 × | 2.9 × | 8.2 × | 4.0 × | 1.2 × | 4.0 × |
| | (W · h/m³) | 10¹⁰ | 10⁷ | 10⁹ | 10⁹ | 10⁸ | 10⁹ | 10⁹ |
| | Rotation energy Q″ | 1.4 × | 4.1 × | 1.9 × | 0.9 × | 2.0 × | 0.6 × | 2.0 × |
| | (W · h/(m³ · kg)) *2 | 10¹⁰ | 10⁷ | 10⁷ | 10⁶ | 10⁸ | 10⁹ | 10⁹ |
| | Treatment flow rate (L/min) | 7 | 7 | 40 | 300 | 7 | 7 | 7 |
| | Number of pass | 1470 | 1 | 1 | 1 | 5.7 | 15 | 50 |
| | Median diameter D₅₀ of | 1.0 | 1.0 | 1.0 | 1.0 | 0.64 | 0.44 | 0.19 |
| | emulsion droplets (μm) | | | | | | | |
| | Reaction rate of silica precursor (%) | 61 | 3 | 5 | 6 | 5 | 4 | 5 |
| Step 2 | Amount of ion exchanged water added | 1192.0 g | 1192.0 g | 89.4 kg | 5384.3 kg | 1192.0 g | 1192.0 g | 1192.0 g |
| | Total amount of oil and silica precursor | 25 | 25 | 25 | 25 | 25 | 25 | 25 |
| | used in step 1 per 100 parts by mass of | | | | | | | |
| | total amount of diluted emulsion liquid | | | | | | | |
| | (part by mass) | | | | | | | |
| | Dilution factor (time) | 1.6 | 1.6 | 1.6 | 1.6 | 1.6 | 1.6 | 1.6 |
| | pH of emulsion liquid after regulating pH | 3.7 | 3.7 | 3.7 | 3.7 | 3.7 | 3.7 | 3.7 |
| | Agitation blade | paddle | paddle | paddle | paddle | paddle | paddle | paddle |
| | | blade | blade | blade | blade | blade | blade | blade |
| | Agitation speed (r/min) | 27 | 27 | 80 | 27 | 27 | 80 | 80 |

| | | | | | | | | |
|---|---|---|---|---|---|---|---|---|
| Oil-containing silica | Median diameter | 2.0 | 2.0 | 2.0 | 2.0 | 1.1 | 1.0 | 0.55 |
| capsule particles | D₅₀ (μm) | | | | | | | |
| | Encapsulation | 33 | 95 | 95 | 95 | 95 | 95 | 90 |
| | rate of oil (%) | | | | | | | |

*1: cetyltrimethylammonium chloride (active ingredient: 30% by mass)
*2: rotation energy Q′ (W · h/m³) per amount (kg) of oil mixture liquid used in step 1

It is understood from Table 2 that the methods of Examples achieve a high encapsulation rate of the oil.

In Examples 1 to 3, 11, and 12, irrespective of the use of a large amount of the oil mixture liquid in the step 1, the period of time required for producing 100 kg of the emulsion liquid is short as described above, from which it is understood that excellent emulsion productivity is achieved.

### Industrial Applicability

The present invention can produce oil-containing silica capsule particles having a high encapsulation rate of the oil. The present invention achieves high emulsion productivity, and therefore is useful as a method of producing oil-containing silica capsule particles in an industrial scale using a large amount of an oil mixture liquid.

1. A method of producing oil-containing silica capsule particles having a core containing an oil and a shell containing silica as a constitutional component, comprising:
step 1: a step of emulsifying an oil mixture liquid containing a surfactant, water, an oil, and a silica precursor, with an in-line emulsifier/disperser, so as to provide an emulsion liquid; and
step 2: a step of forming oil-containing silica capsule particles by using the emulsion liquid obtained in the step 1, in a batch type agitation tank.

2. The method of producing oil-containing silica capsule particles according to claim 1, wherein the emulsion liquid obtained in the step 1 has a reaction rate of the silica precursor of 80% or less.

3. The method of producing oil-containing silica capsule particles according to claim 1 or 2, wherein the in-line emulsifier/disperser used in the step 1 has a rotor and a stator.

4. The method of producing oil-containing silica capsule particles according to claim 3, wherein in the emulsification in the step 1, rotation energy Q″ of the rotor per a capacity of an emulsifying/dispersing chamber of the in-line emulsifier/disperser and an amount of the oil dispersion liquid is 1×10⁴ W·h/(m³·kg) or more.

5. The method of producing oil-containing silica capsule particles according to any one of claims 1 to 4, wherein an amount of the oil mixture liquid used in the step 1 is 100 kg or more.

6. The method of producing oil-containing silica capsule particles according to any one of claims 1 to 5, wherein an emulsification time of the step 1 is 12 hours or less.

7. The method of producing oil-containing silica capsule particles according to any one of claims 1 to 6, wherein the silica precursor contains a tetraalkoxysilane.

8. The method of producing oil-containing silica capsule particles according to any one of claims 1 to 7, wherein in the step 2, a pH modifier is added to the emulsion liquid obtained in the step 1 to regulate pH of the emulsion liquid to 4.5 or less, and then the oil-containing silica capsule particles are formed in the batch type agitation tank.

9. The method of producing oil-containing silica capsule particles according to any one of claims 1 to 8, wherein in the step 2, the emulsion liquid obtained in the step 1 is diluted by adding water thereto, and then the oil-containing silica capsule particles are formed in the batch type agitation tank.

10. The method of producing oil-containing silica capsule particles according to claim 9, wherein the dilution in the step 2 is performed to make a total amount of the oil and the silica precursor used in the step 1 of 35 parts by mass or less per 100 parts by mass of the emulsion liquid after the dilution.

11. The method of producing oil-containing silica capsule particles according to any one of claims 1 to 10, wherein a mass ratio of the water phase component containing the surfactant and water and the oil phase component containing the oil and the silica precursor of the oil mixture liquid used in the step 1 (water phase component/oil phase component) is 50/50 or more and 99/1 or less.

12. The method of producing oil-containing silica capsule particles according to any one of claims 1 to 11, wherein the emulsification in the step 1 is performed by using the in-line emulsifier/disperser by an external circulation system.

13. The method of producing oil-containing silica capsule particles according to any one of claims 1 to 11, wherein the emulsification in the step 1 is performed in such a manner that the surfactant, water, the oil, and the silica precursor are mixed in in-line mode to prepare the oil mixture liquid, and then the oil mixture liquid is supplied to the in-line emulsifier/disperser.

14. The method of producing oil-containing silica capsule particles according to any one of claims 1 to 13, wherein a number of pass of the in-line emulsifier/disperser in the step 1 is 1 or more.

15. The method of producing oil-containing silica capsule particles according to any one of claims 1 to 14, wherein emulsion droplets of the emulsion liquid obtained in the step 1 have a median diameter D₅₀ of 10 μm or less.

16. The method of producing oil-containing silica capsule particles according to any one of claims 1 to 15, wherein in the step 2, a disperser using an agitation blade is used.

17. The method of producing oil-containing silica capsule particles according to any one of claims 1 to 16, wherein the emulsion liquid obtained in the step 1 has a reaction rate of the silica precursor of 55% or less.
